(19) United States
(12) Patent Application Publication
(10) Pub. No.: US 2016/0045622 A1
(43) Pub. Date: Feb. 18, 2016

(54) IN VIVO CELL IMAGING

(71) Applicant: ABETERNO LIMITED, Cambridge (GB)

(72) Inventor: Rosalind Codrington, Impington, Cambridgeshire (GB)

(73) Assignee: ABETERNO LIMITED, Cambridge (GB)

(21) Appl. No.: 14/783,180

(22) PCT Filed: Mar. 20, 2014
(86) PCT No.: PCT/GB2014/050872

§ 371 Date: Oct. 8, 2015

(30) Foreign Application Priority Data
Apr. 11, 2013 (GB) 1306589.1

(51) Int. Cl. A61K 49/00; G01N 33/58

(52) U.S. Cl. CPC A61K 49/0058; A61K 49/0056; G01N 33/582

(57) ABSTRACT

This disclosure relates to the live imaging of native intracellular proteins in eukaryotic cells, typically mammalian cells, by means of a fluorescent bead coupled to a ligand, a cell penetrating peptide and a cell export peptide adapted to interact with cell secretory proteins to remove non-bound fluorescent beads.

### INTRODUCTION

This disclosure relates to the live imaging of native intracellular proteins in eukaryotic cells, typically mammalian cells, by means of a fluorescent bead coupled to a ligand, a cell penetrating peptide and a cell export peptide to facilitate removal of fluorescent beads that are not bound to native intracellular protein. Optionally the cell penetrating peptide and a cell export functions may reside in a single peptide.

### BACKGROUND

Live cell imaging (LCI) is of great importance in biochemistry and cell biology. The approach has provided novel and exciting insights through the observation and understanding of the intracellular events that govern the various biological phenomena. It can provide information on a range of molecular processes and reactions with very high specificity and sensitivity, through the use of state-of-the-art microscopy and computer vision techniques (Tokuko, 2002). The probes are a key element to LCI and interact with specific targets such as proteins, nucleic acids, ROS (Reactive Oxygen Species), organelles, cell membranes, etc (Patterson, 2007). The most commonly used probes are the genetically encoded markers such as GFP and over 35 of its variants (Tsien, 1998; Patterson, 2007; Chudakov et al., 2010). These can be inserted into the genome and used to selectively label specific targets and cellular compartments (Tsien, 1998; Patterson, 2007). However, there are whole ranges of fluorescent indicators that can be either inserted into the genome or conjugated to specific cellular targets, and with these probes we have seen as well the evolution of advanced microscopy techniques such as FRET (Fluorescent Resonance Energy Transfer) (Mao et al., 2008; Cho et al., 2013), FLIM (Fluorescence Lifetime Imaging Microscopy) (Treanor et al., 2005), FCS (Fluorescent Correlation Microscopy) (Bacia et al., 2008), BiFC (bimolecular fluorescence complementation assay) (Kerppola, 2006), or 2PE (Two-Photon Excitation Fluorescent Microscopy) (Zipfel et al., 2003). Nevertheless, fluorescent proteins have the downside that they are large in size (˜27 kDa) and can therefore interfere with the natural behaviour of the protein of interest. Chemical tags, on the other hand are relatively small in size and are not as prone to interfering with the function of the target protein (Liu et al., 2012). However, they rely on the interaction between a genetically encoded tag on the protein of interest and a small molecule that can render the target fluorescent (Liu et al., 2012; Wombacher & Cornish, 2011, Watkins et al., 2009; Uttamapinant et al., 2010). Therefore, a downside of these fluorescent probes is that they require the host cell to be genetically modified, thereby requiring complex cloning procedures and cell delivery strategies.

Fluorescently labelled monoclonal antibodies (mAb) are widely used in cell imaging; however they require cell fixation and membrane permeabilization, due to the inherent difficulties surrounding cell internalisation of antibodies. Furthermore, whole monoclonal antibodies cannot be expressed in cells due to the reducing environment of the cell. This has made it difficult so far to use mAbs for intracellularly expressed protein targets in LCI. However, antibody fragments such as single VH or VL domains are routinely stably expressed in cells and have been internalised into cells with the help of carrier mediated systems such as transduction peptides (Futaki et al., 2001; Avignolo et al., 2008; Niesner et al., 2002; Ma et al., 2011).

Inorganic nanomaterials such as quantum dots (QDs) are known in the art. They remove the need for generating genetically modified cells for the expression of fluorescent proteins and chemical tags and have superior photostability and quantum efficiency over their organic counterparts (Michalet et al., 2005; Walling et al., 2009). These are semiconductor nanocrystals, most frequently composed of a CdSe core with a ZnS shell (Liu et al., 2008), and an outer hydrophilic coating made of functional ligands that allow them to bind to biomolecules (Liu et al., 2008; Michalet et al., 2005; Chen et al., 2010; Walling et al., 2009). They offer unique optical properties, such as resistance to bleaching (Rhyner et al., 2008), enhanced signal brightness due to larger extinction coefficients than most other dyes (Chan et al., 2002), size-tunable light emission (Alivisators, 1996), and simultaneous excitation of multiple fluorescence colors (Gao et al., 2004).

Despite advancements in probes and bio-imaging techniques in LCI, the current methods for labelling intracellular proteins remain a challenge. Particularly, efforts to visualise specific intracellular proteins can be hampered due to non-specific binding, problems with intracellular delivery (Michalet et al., 2005), and a high non-specific background in the cytoplasm due to the target protein not being present. Furthermore, multi-coloured fluorescent labelling on intracellular and extracellular targets remains challenging. It often requires a combination of techniques that makes use of different targeting probes, intricate cloning procedures, various cell delivery strategies (e.g. peptide-mediated cell transductions, transfection reagents, electroporation or microinjection), membrane permeabilisation via cellular fixation, multi-step bioconjugation chemistries and complex spectral de-convolution.

Recent studies have reported to have successfully delivered QDs into cells by conjugating these to a cell penetrating peptide and an antibody fragment specific to the target protein (Michalet et al., 2005). However, if the antibody moiety is not present on the QD it results in a non-specific accumulation of QDs in the cytoplasm. Conversely, the lack of expression or low expression levels of the target protein will result in poor targeting abilities by the QDs causing an accumulation of the nanoparticles in the cytoplasm and ultimately in a background. There are currently no means by which the nanoparticles can exit the cells if the target proteins are not present. Therefore, most studies require genetically modified cells to ensure the expression of their target protein of interest to remove background.

The current bioimaging techniques for LCI still require either cellular fixation or genetically modified cells to ensure the expression of the target proteins, to generate chemical tags and fluorescent proteins. The present disclosure is directed to overcoming the deficiencies in live cell imaging of intracellular proteins by providing a method that will remove the need for genetically modified cells and remove the background resulting from non-expression of the target protein of interest.

### STATEMENTS OF INVENTION

According to an aspect of the invention there is provided a fluorescent bead comprising: a ligand that specifically binds an intracellular antigen, a cell membrane penetrating cationic peptide and a peptide comprising an amino acid sequence that is adapted to interact with the secretory pathway in a cell.

Fluorescent particles include semiconductor Nanoparticles (NPs) (Quantum Dots), metal NPs, silica NPs and other polymer NPs. Optionally the cell penetrating peptide and a cell export functions may reside in a single peptide.

In a preferred embodiment of the invention said fluorescent bead comprises a magnetic particle.

In an alternative preferred embodiment of the invention said fluorescent bead comprises a quantum dot.

Quantum dots (QDs) are nanometer-sized semiconductor particles with fluorescent optical properties that can be adjusted by their chemical composition, size, or shape. QDs are commonly used for biological and biomedical applications, such as diagnostics, bio-sensing and bio-labelling. QD are made typically from inorganic semiconductor nanocrystals with a diameter of between 2-8 nm and have luminescent properties. QD are generally composed of atoms from groups II and VI elements (e.g. CdSe and CdTe) or groups III and V elements (e.g. InP and InAs) of the periodic table. The most commonly used QD system comprises an inner semiconductor core of CdSe coated with the outer shell of ZnS. QDs can be made to emit fluorescent light in the ultraviolet to infrared spectrum just by varying their size.

Quantum dot particles comprise chalcogens (such as sulphur, selenium, tellurium) and transition metals like cadmium or zinc. Conductivity of quantum dots can be increased by adding further metals such as thulium and rhenium. This process is known as doping. Doping can also introduce manganese into the quantum dot to provide it with magnetic properties. Quantum dot metals can be toxic to biological systems and suffer from low solubility, therefore, biocompatible polymers are widely used to cover and coat the quantum dots. Examples of polymeric materials are dextran, PEG, thiol terminated OH-poly(amidoamine), chitosan, poly(acryloyoxysuccinimide), poly(2-(dimethy-lamino)ethyl methacrylate), linear poly(allylamine) (PAL), hyperbranched poly(ethyleneimine) (PEI), poly(N-isopropylacrylamide) and poly(N-vinylcaprolactam). Polymers can carry also ionic or functional anchor groups, such as thiol, amine and carboxyl-groups.

Biomolecules such as proteins and antibodies can be conjugated to quantum dots via covalent and non-covalent linkage. For example, covalent binding can be achieved by using crosslinking agents such as glutaraldehyde or 1-Ethyl-3-[3-dimethylaminopropyl]carbodiimide hydrochloride coupling functional groups such as —COOH, —NH2 or —SH present on the QD surface to the functional groups present on the biomolecules. Methods and agents to facilitate cross linking are known in the art and described by Duane E. Prasuhn, Kimihiro Susumu, Igor L. Medintz. In Biomedical Nanotechnology: Multivalent Conjugation of Peptides, Proteins, and DNA to Semiconductor Quantum Dots; Methods in Molecular Biology, Volume 726, 2011, pp 95-110. Further methods to crosslink biological molecules to quantum dots are disclosed in patent application WO02/055186, WO2012/29443, WO2011/056575 and US2010/05089 and are hereby incorporated by reference in their entirety.

Various covalent and non-covalent strategies have been developed for conjugating biomolecules such as proteins and antibodies to the QDs. Biomolecules can be bound covalently using functional groups such as —COOH, —NH2 or —SH present on the QD surface. One strategy employs N-ethyl-N′-(3-diethylaminopropyl) carbodiimide (EDC) as a heterocrosslinker, which crosslinks the carboxylate group of the QDs to the amine group of a protein. This method does not require any chemical modification of the protein as most proteins contain a primary amine.

An alternate strategy is based on the active ester maleimide-mediated coupling of amine and sulfhydryl groups. Others employ pre-activated amphiphilic polymers containing multiple anhydride units, which are highly reactive towards primary amines, for binding protein to QDs. Coating of QD with molecules such as streptavidin allows the conjugation of biotinylated biomolecules to QD. Self-illuminating QD conjugates have also been developed by conjugating luciferase (Luc8) to polymer-coated CdSe/ZnS core shell QD. Moreover, antibodies against cancer biomarkers were bound to the QD-Luc8 complex using a 1-ethyl-3-(3-dimethylaminopropyl) carbodiimide hydrochloride crosslinker.

Non-covalent associations have been used to bind immunoglobulins [IgG] to QDs by using a positively charged leucine zipper domain that binds electrostatically to the negatively charged QDs and a protein G domain that binds to the constant Fc region of IgG thus leaving the F(ab)2 region free for antigen binding. A technique based on the targeting of Ni-nitriloacetic acid moieties against hexa-histidine motifs, as employed in case of dyes, is utilised for binding hexa-histidine-tagged biomolecules to QDs using nickel-nitrilotriacetic acid (Ni-NTA) as the chelating agent. It is known in the art to modify CdSe QD with an impermeable coating of a polymer that prevents the leakage of highly toxic cadmium ions from the QD conjugate and provided a means to chemically attach tumour-targeting molecules and drug delivery functionality to the QD conjugate. U.S. Pat. No. 8,378,075 and U.S. Pat. No. 6,326,144, each of which is incorporated by reference, disclose methods allowing the association and/or direct attachment of proteins or peptides onto the surface of QD.

Besides QDs, highly fluorescent nanoparticles comprising functional groups can also be used to conjugate functional molecules such as peptides or protein. PAN (polyacrylonitrile) particles are ideally suited for FRET applications. They are highly fluorescent and extremely small (less than 30 nm in diameter). They have low interference by non-specific adsorption or other interactions. A high density of carboxyl groups on their surface allows covalent coupling of biomolecules. Other nanoparticles are silica based doped with a fluorescent dye. US2013/0266957, which is incorporated by reference, discloses fluorescent nanoparticles with a dimension of up to 1000 nm formed from poly[2-methoxy-5-(2-ethylhexyloxy)-1,4-(1-cyanovinylene-1,4-phenylene) comprising functional groups.

In a preferred embodiment of the invention said fluorescent bead comprises two or more fluorescent molecules wherein said molecules have different excitation/emission wavelengths.

In a preferred embodiment of the invention said intracellular antigen is substantially a nuclear antigen.

In a preferred embodiment of the invention said nuclear antigen is selected from the group consisting of: FoxO family members, TARP, p53, Rb, E2F, hK4, BRCA1, Cdk2, SATB1, TP53INP1, Myc, Fos, Jun, CREB, Ets, SRF, FAK, Pax6, Calpain, Elk1, Stats 1-3, Akt, p21.

In an alternative preferred embodiment of the invention said intracellular antigen is substantially a cytoplasmic antigen.

In a preferred embodiment of the invention said cytoplasmic antigen is selected from the group consisting of: Jak1, Jak2, Tyk2, Jak3, GATA 1-4, Stats 1-6, CBP, NFkB, IKK, PIK3CA, B-raf, EBI3, elF2α, Akt, PI3K, IAP, Hsp, FAK, Raf, Ras, TNF, Src, Abl, Caspases 1-12.

In a preferred embodiment of the invention said cationic cell membrane penetrating peptide is a natural cell penetrating peptide.

In an alternative preferred embodiment of the invention said cell membrane penetrating peptide is a synthetic sequence.

In a preferred embodiment of the invention said cell membrane penetrating peptide is adapted to penetrate a eukaryote cell, preferably a mammalian cell

In a preferred embodiment of the invention said cell membrane penetrating peptide is selected from the group consisting of: YARKKRRQRRR (SEQ ID NO: 1), YARKARRQARR (SEQ ID NO: 2), YARAAARQARA (SEQ ID NO: 3), YARAARRAARR (SEQ ID NO: 4), YARAARRAARA (SEQ ID NO: 5), YARRRRRRRRR (SEQ ID NO: 6), YAAARRRRRRR (SEQ ID NO: 7), PLSSIFSRIGDP (SEQ ID NO: 8), RQIKIWFQNRRMKWKK (SEQ ID NO: 9), WEIEDEDER (SEQ ID NO: 10), GRKKRRQRRRPQ (SEQ ID NO: 11), RRRRRRRRRRRR (SEQ ID NO:12) or GRKKRRQRRRPQC (SEQ ID NO: 20).

In a preferred embodiment of the invention said cell membrane penetrating peptide consists essentially of the amino acid sequence: GRKKRRQRRRPQ (SEQ ID NO: 11) or GRKKRRQRRRPQC (SEQ ID NO: 20).

Preferably said cell membrane penetrating peptide consists of the amino acid sequence: GRKKRRQRRRPQ (SEQ ID NO: 11) or GRKKRRQRRRPQC (SEQ ID NO: 20).

In a preferred embodiment of the invention said cell membrane penetrating peptide consists essentially of the amino acid sequence: RRRRRRRRRRRR (SEQ ID NO: 12).

Preferably said cell membrane penetrating peptide consists of the amino acid sequence: RRRRRRRRRRRR (SEQ ID NO: 12).

The cell penetrating peptide and the secretory peptide can be engrafted into the CDR3 regions of the VL and VH domains.

In a preferred embodiment of the invention there is provided a single peptide comprising a cell penetrating domain and a domain adapted to facilitate, when in use, the removal of fluorescent beads that are not bound to said native intracellular protein.

Preferably said peptide comprises the amino acid motif RXRR or RXKR where X is any amino acid residue.

In a preferred embodiment of the invention said peptide adapted to interact with the eukaryote secretory pathway is selected from the group consisting of:

(SEQ ID NO: 13)
MCPARSLLLVATLVLLDHLSLA;
(SEQ ID NO: 14)
MDAMKRGLCCVLLLCGAVFVSPS;
(SEQ ID NO: 15)
MATGSRTSLLLAFGLLCLPWLQEGSA;
(SEQ ID NO: 16)
MYRMQLLSCIALSLALVTNS;
(SEQ ID NO: 17)
MGVKVLFALICIAVAE;
(SEQ ID: 19)
MGVKVLFALICIAVAEA;
or
(SEQ ID NO: 21)
MGVKVLFALICIAVAEAC.

In a preferred embodiment of the invention said peptide is adapted to interact with the mammalian secretory pathway.

In a preferred embodiment of the invention said peptide is consists essentially of the amino acid sequence MGVKVLFALICIAVAE (SEQ ID NO: 17), MGVKVLFALICIAVAEA (SEQ ID: 19) or MGVKVLFALICIAVAEAC (SEQ ID NO: 21).

In a preferred embodiment of the invention said peptide is consists of the amino acid sequence: MGVKVLFALICIAVAE (SEQ ID NO: 17), MGVKVLFALICIAVAEA (SEQ ID: 19) or MGVKVLFALICIAVAEAC (SEQ ID NO: 21).

In a preferred embodiment of the invention said peptide is consists essentially of the amino acid sequence: MYRMQLLSCIALSLALVTNS (SEQ ID NO: 16).

In a preferred embodiment of the invention said peptide is consists of the amino acid sequence: MYRMQLLSCIALSLALVTNS (SEQ ID NO: 16).

In a further preferred embodiment of the invention said bead further comprises a nuclear export peptide.

In a preferred embodiment of the invention said nuclear export peptide comprises or consists essentially of the amino acid sequence: NELALKLAGLDINKTEGEEDAQ [SEQ ID NO: 18].

Peptides according to the invention are typically at least 10 amino acids in length but can be 11, 12, 13, 14, 15, 16, 17, 18, 19, 20, 25, 30, 35, 40, 45 or at least 50 amino acids in length.

In a preferred embodiment of the invention said ligand that binds said intracellular antigen is an antibody fragment.

Various fragments of antibodies are known in the art, e.g. Fab, Fab2, F(ab′)2, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab2 fragment comprises two joined Fab fragments. When these two fragments are joined by the immunoglobulin hinge region, a F(ab′)2 fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as William E. Paul, Fundamental Immunology or Janeway et al. Immunobiology (cited above). Molecular biology now allows direct synthesis (via expression in cells or chemically) of these fragments, as well as synthesis of combinations thereof. A fragment of an antibody or immunoglobulin can also have bispecific function as described above.

Methods to deliver antibody fragments to cells intracellularly are known in the art; for example see WO2007/064727; WO2004/030610; WO03/095641; WO02/07671; WO01/43778; WO96/40248; and WO94/01131 each of which is incorporated by reference in their entirety.

In a preferred embodiment of the invention said antibody fragment is a ScFv or a single VH or VL domain.

In an alternative preferred embodiment of the invention said ligand that binds said intracellular antigen is a peptide or a peptide aptamer or a nucleic acid aptamer.

Peptides that have binding affinity to a target antigen are within the scope of the invention. For example peptides that mimic the binding affinity of antibodies and antibody fragments; see Chattophadayay, A, Tate, S., Beswick, R., Wagner, S. D. and Ko Ferrigno, P. A peptide aptamer to antagonise BCL-6 function. Oncogene, 25 2223-2233 (2006). Nucleic acid aptamers that have binding affinity to a target antigen are within the scope of the invention. For example of RNA aptamers that can mimic the binding affinity of antibodies and antibody fragments see Ellington A D, Szostak J W (August 1990). “In vitro selection of RNA molecules that bind specific ligands”. Nature 346 (6287): 818-22.

In a further alternative embodiment of the invention said intracellular antigen is a transcription factor and said ligand is a nucleic acid comprising a nucleotide binding motif for said transcription factor.

In a preferred embodiment of the invention said transcription factor is selected from the group consisting of: TEF-1, CREB, RUNX3, NFκB, YAP-1, HUR, HNF3 [FOXA], Hox-1.3, STAT or p53.

In a preferred embodiment of the invention said nucleotide binding motif is selected from the sequences set forth in SEQ ID: NO 24, 25, 26, 27, 28, 29, 30, 31, 32, 33, 35, 36 or 37.

According to a further aspect of the invention there is provided a fluorescent bead according to the invention for use in live imaging of eukaryotic cells.

In a preferred embodiment of the invention said eukaryotic cells are mammalian cells.

In a preferred embodiment of the invention said mammalian cells are human.

In a preferred embodiment of the invention said eukaryotic cells are stem cells.

In a preferred embodiment of the invention said stem cells are pluripotent stem cells.

In an alternative preferred embodiment of the invention said stem cells are multipotent.

In a still further preferred embodiment of the invention said eukaryotic cells are lineage restricted progenitor cells.

The term “stem cell” represents a generic group of undifferentiated cells that possess the capacity for self-renewal while retaining varying potentials to form differentiated cells and tissues. Stem cells can be pluripotent or multipotent. A pluripotent stem cell is a cell that has the ability to form all tissues found in an intact organism although the pluripotent stem cell cannot form an intact organism. Furthermore, it is known that human somatic cells can be re-programmed to an undifferentiated state similar to an embryonic stem cell. For example, WO2007/069666 describes re-programming of differentiated cells (e.g. mouse fibroblast cells) without the need to use embryonic stem cells. Nuclear re-programming is achieved by transfection of retroviral vectors into somatic cells that encode nuclear re-programming factors, for example Oct family, Sox family, Klf family and Myc family of transcription factors. The somatic cells de-differentiate and express markers of human embryonic stem cells to produce an “induced pluripotent cell” [iPS]. In Takahashi et al [Cell vol 131, p 861-872, 2007] adult human dermal fibroblasts with the four transcription factors: Oct3/4, Sox2, Klf4, and c-Myc de-differentiate to human ES cells in morphology, proliferation, surface antigens, gene expression, epigenetic status of pluripotent cell-specific genes and telomerase activity.

A multipotent cell has a restricted ability to form differentiated cells and tissues. Typically, adult stem cells are multipotent stem cells and are the precursor stem cells or lineage restricted stem cells that have the ability to form some cells or tissues and replenish senescing or damaged cells/tissues. Generally they cannot form all tissues found in an organism, although some reports have claimed a greater potential for such ‘adult’ stem cells than originally thought. Examples of multipotent stem cells include mesenchymal stem cells. Mesenchymal stem cells differentiate into a variety of cell types that include osteoblasts, chondrocytes, myocytes, adipocytes and neurones. Typically, mesenchymal stem cells are obtained from bone marrow. Currently, stem cell therapies are exploring different sources of pluripotent and multipotent stem cells and cell culture conditions to efficiently differentiate stem cells into cells and tissues suitable for use in tissue repair.

In an alternative preferred embodiment of the invention said stem cell is a cancer stem cell.

The concept of a cancer stem cell within a more differentiated tumour mass, as an aberrant form of normal differentiation, is now gaining acceptance over the current stochastic model of oncogenesis, in which all tumour cells are equivalent both in growth and tumour-initiating capacity (Hamburger et al., 1977; Pardal et al., 2003). For example, in leukaemia, the ability to initiate new tumour growth resides in a rare phenotypically distinct subset of tumour cells (Bonnet et al., 1997) which are defined by the expression of CD34+CD38 surface antigens and have been termed leukemic stem cells (LSC). Similar tumour-initiating cells have also been found in ‘solid’ cancers such as prostate (Collins et al., 2005), breast (AlHajj et al., 2003), brain (Singh et al., 2004), lung (Kim et al., 2005), colon (O'Brien et al., 2007; Ricci Vitiani et al., 2007) and gastric cancers (Houghton et al., 2004). A list of intracellular cancer stem cell markers is available in Table 1.

According to a further aspect of the invention there is provided a method to image live eukaryotic cells, preferably mammalian cells comprising the steps:
i) providing a cell sample contacting said eukaryotic cell or mammalian cell with a fluorescent bead according to the invention;
ii) allowing uptake of said fluorescent bead by the cell and binding to an intracellular target; and
iii) applying electromagnetic radiation of a defined wavelength to detect the intracellular target thereby imaging said cell.

The fluorescent bead may be coupled to an antibody fragment (scFv, single domain, dibody), a cell penetrating peptide and a secretory peptide, or to an antibody fragment and a secretory peptide. The cell penetrating peptide and the secretory peptide may be either directly coupled to the fluorescent bead or to the antibody fragment via expression as a fusion protein or via chemical coupling between the antibody and the peptide, or they may be engrafted within the CDR3 regions of the VH or VL domains as previously described by (Jeong et al. 2011).

In a preferred method of the invention said mammalian cells are human.

The crosslinked-antibody fragments are incubated with the cells and allowed to internalize into the cells. After achieving cell internalisation of the crosslinked fluorescent bead the cells are allowed a period of incubation in cell media to allow binding of the fluorescent beads to their targets. Any unbound particles will then be secreted from the cells by the secretory peptide.

According to an aspect of the invention there is provided a fluorescent bead for use in in vivo eukaryotic cell imaging comprising: a ligand that specifically binds an intracellular antigen, a peptide comprising a cell penetrating domain and a domain adapted to facilitate removal of fluorescent beads that are not bound to native intracellular protein.

In this aspect of the invention cell penetration and the removal of unbound beads is provided by a single peptide comprising a cell penetration function and a function that allows the bead to interact with the secretory pathway to remove unbound beads from the cell.

In a preferred embodiment of the invention said cell penetrating domain comprises one or more positively charged amino acid residues.

In a preferred embodiment of the invention said positively charged amino acid residues comprise one or more amino acids selected from the group consisting of: arginine, lysine and histidine.

Preferably said peptide comprises the amino acid motif RXRR or RXKR where X is any amino acid.

In a preferred embodiment of the invention said peptide comprises or consists of the amino acid sequence: GRKKRRQRRRPQ [SEQ ID NO: 11] or GRKKRRQRRRPQC (SEQ ID NO: 20).

In a further preferred embodiment of the invention said bead further comprises a nuclear export peptide.

In a preferred embodiment of the invention said nuclear export peptide comprises or consists essentially of the amino acid sequence: NELALKLAGLDINKTEGEEDAQ [SEQ ID NO: 18].

According to a further aspect of the invention there is provided the use of a fluorescent bead in in vivo cell imaging.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

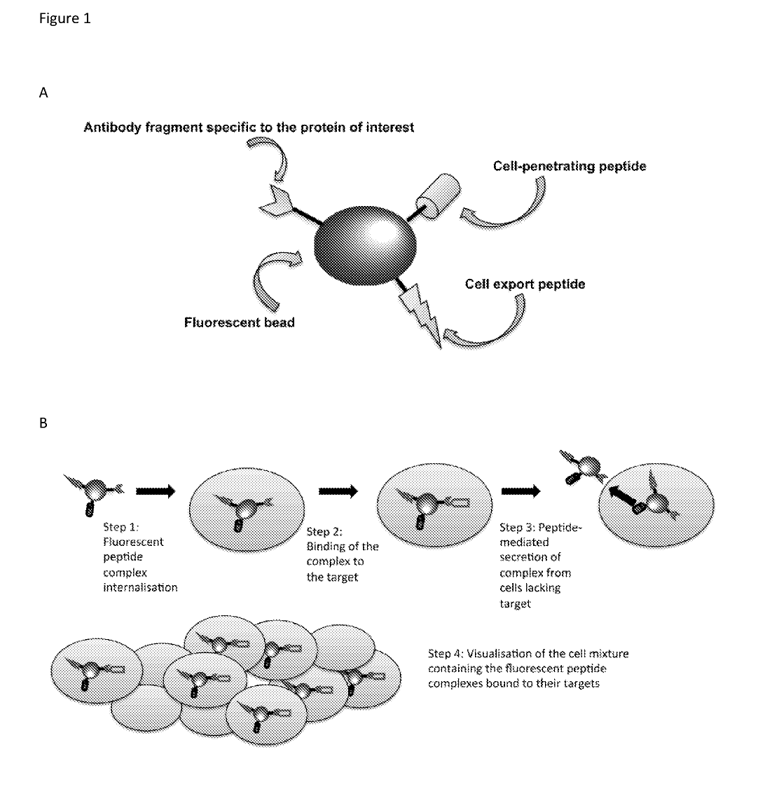

FIG. 1: Diagram illustrating the principle of the invention. (A) A fluorescent particle coupled to a cell penetrating peptide, a cell export peptide and an scFv antibody fragment. (B) Step 1. The fluorescent peptide complex is incubated with the mixed cell population for a length of time until the complex has fully internalised into the cells. Step 2. The scFv antibody fragments specifically bind to their antigen targets in the cells and are thereby anchored inside these cells. Step 3. The fluorescent peptide complexes that did not bind to their specific target antigens are removed from the cells via the cell secretory pathway. This step avoids the non-specific accumulation of the fluorescent nanoparticles in cells that do not express the specific protein target of interest. Step 4. The cells can now be visualised under a fluorescent microscope;

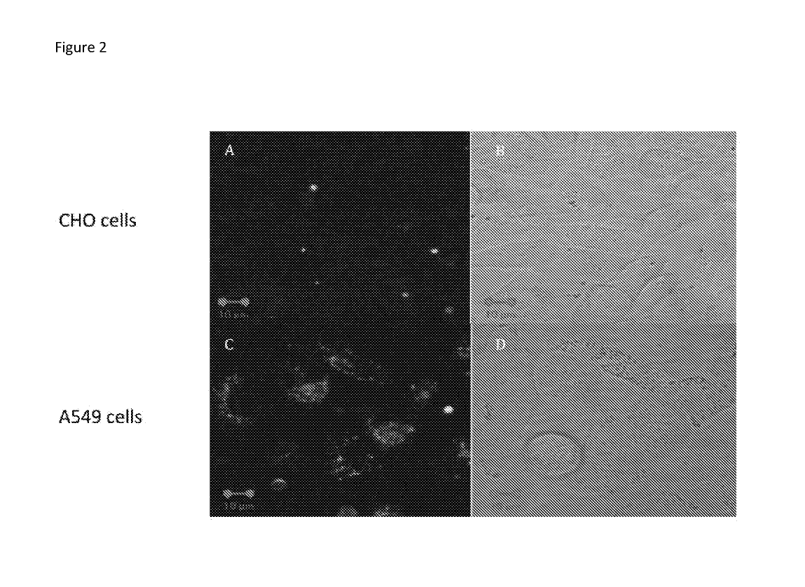

FIG. 2: Visualisation of the green fluorescent peptide particles in CHO cells and A549 cells. Confocal images of CHO (A, B) and A549 (C, D) cells after 4.30 h incubation with the scFv magnetic-protein complexes. The cells were washed vigorously with PBS before imaging. The green magnetic-protein complexes localised at the cell membrane, the perinuclear membrane and in the cytoplasm consistent with the sub-cellular localisation of activated Ras in the A549 lung cancer cell line. No magnetic-protein complexes were observed in CHO cells following 4.30 h incubation;

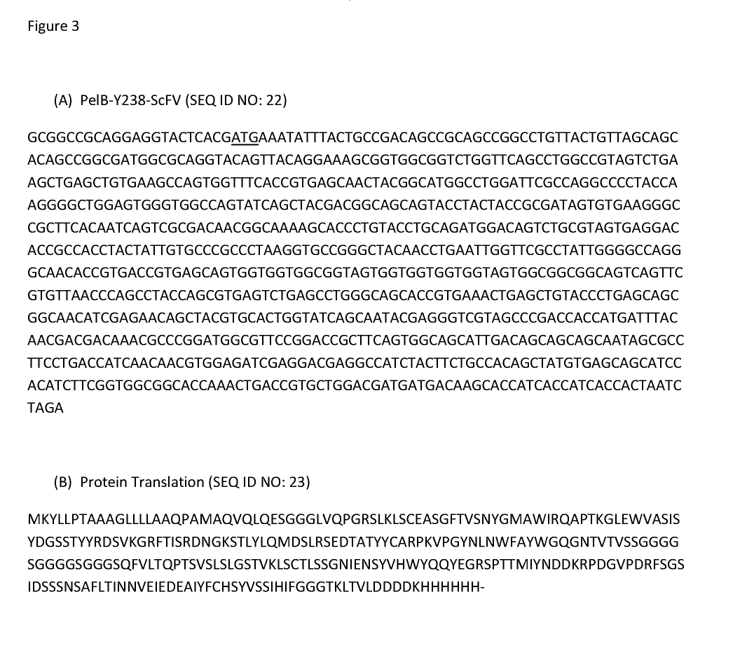

FIG. 3 (A) PelB-Y238-ScFV nucleotide sequence (SEQ ID NO: 22). The start codon is underlined. (B) Translated amino acid sequence (SEQ ID NO: 23); and

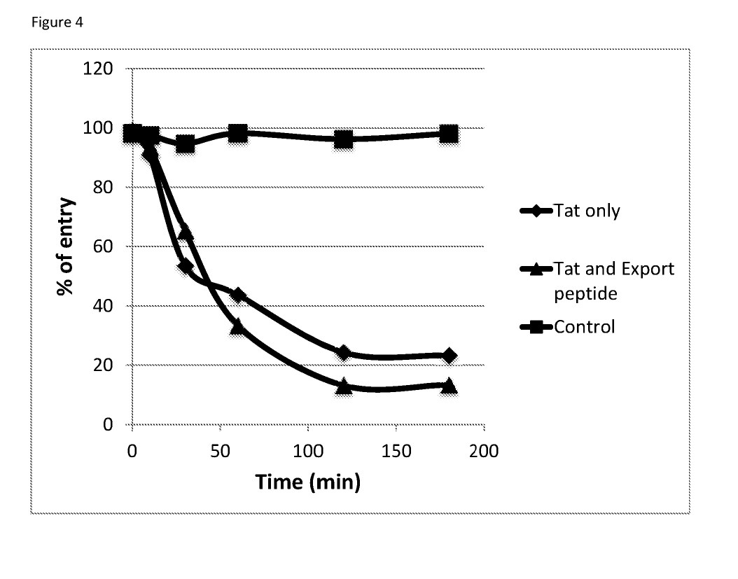

FIG. 4 Effects of magnetic particles loaded with 1,000 Tat peptides per bead (Tat only, closed diamond) and 1,000 Tat peptides and 10 export peptides per bead (Tat and Export peptide, closed triangle). Peptides were incubated with CHO cells at 10¹¹ per well (with 1 million cells per well). Controls (closed squares) are beads functionalised with poly dimethylamine and with no peptides attached. Functionalised bead enter the cells and remain trapped inside the cell throughout the experimental period (100% entry). Beads with Tat peptide alone were retained in the cells to 24.3% after 120 min incubation; whereas beads with the Tat peptide and export peptide were retained in the cell to just 13.2% during the same time period.

### MATERIALS AND METHODS

Production of H-RasG12V scFv

The sequence of an scFv (scFv#6) specific for the mutant H-RasG12V (Tanaka et al., EMBO Journal 2007) served as a template for the synthesis of scFv#6 by GeneArt® (Life Technologies) with flanking BamH1 and EcoR1 sites at the 5′ and 3′ ends, respectively. The gene was subcloned into BamH1 and EcoR1 sites of the pRSETa bacterial expression vector (Life Technologies) to generate in-frame fusion scFv protein with a 6× histidine tag. The plasmid was transformed into BL21(DE3) bacterial cells (Merck-Millipore) and expression of the scFv#6 fragment was induced in 500 ml culture via the addition of 1 mM IPTG at OD₆₀₀ of 0.55 followed by incubation for 3 h at 37° C. The cells were harvested by centrifugation at 5000×g at 4° C. and the final cell pellets were stored at −80° C. prior to scFv purification.

scFv protein was extracted from bacterial pellets in 25 ml of B-PER bacterial lysis buffer (Thermo Scientific) in the presence of a cocktail of protease inhibitors (containing 1.5 μg/ml Chymotrypsin, 0.8 μg/ml Thermolysin, 1 mg/ml Papain, 1.5 μg/ml Pronase, 1.5 μg/ml Pancreatic extract, 0.002 μg/ml Trypsin) (Roche) at RT for 30 min. The lysate was centrifuged at 15000×g at 4° C. and the supernatant was diluted in 25 ml lysis buffer (50 mM Na phosphate, pH 8.5, 300 mM NaCl, 10 mM imidazole). The His-tagged scFv#6 proteins were purified by gravity flow through 1 ml of Ni-NTA agarose (Qiagen), followed by a wash with 20 ml of wash buffer (50 mM Na phosphate, pH 8.5, 300 mM NaCl, 20 mM imidazole) and eluted in 5 ml of elution buffer (50 mM Na phosphate, pH 8.5, 300 mM NaCl, 250 mM imidazole). The eluted protein was subsequently dialysed against 0.1M MES pH6.0, snap frozen and stored at −80° C. at a concentration of 1.4 mg/ml

Production of Anti-H-Ras scFv Y238

The sequence of an scFv (Y238) specific for H-Ras (O. Cochet et al, Molecular Immunology (35) 1998) served as a template for the synthesis of the Y238 gene containing a PelB leader sequence on the 5′ end to generate PelB-Y238 (Generon Ltd) with flanking Not1 and Xba1 sites at the 5′ and 3′ ends, respectively. The gene was subcloned into Not1 and Xba1 sites of the pSF-T7/LacO bacterial expression vector to generate in-frame fusion scFv protein with a 6× histidine tag (Generon Ltd). The plasmid was transformed into Lemo21 (DE3) bacterial cells and expression of the Y238 scFv fragment was induced in a 6 L culture via the addition of 400 μM IPTG at OD600 of 0.55 followed by incubation for 16 h at 25° C. The periplasmic fraction was isolated by osmotic shock, applied to Ni NTA (ProBound, Invitrogen) for 90 mins at room temperature and eluted with 300 mM imidazole (Generon Ltd). The eluted protein was subsequently dialysed against PBS pH7.4, before coupling to the nanoparticles.

Nanoparticles

Magnetic nanoparticles with an average size of 100 nm were purchased from Chemicell, Berlin, Germany. These particles are referred to as nano-screenMAG particles with an extension to their name -ARA, referring to the surface coating of the iron oxide core with Glucuronic acid used for the binding to biomolecules, and are covered by a lipophilic fluorescence dye emitting light in the range of green (Exc.=476 nm/Em_{max.}=490 nm). For coupling to the carboxyl function of protected peptides the “-PEA” variant of the particles was used, with this suffix referring to a polydimethylamine coating of the surface of the iron oxide core.

Coupling of the Fluorescent Nanoparticles to Proteins

FITC labelled Tat cell penetrating peptide 5(6)-Carboxyfluorescein-GRKKRRQRRRPQ (SEQ ID 11) and Gaussia Luciferase secretory peptide 5(6)-Carboxyfluorescein-MGVKVLFALICIAVAEA (SEQ ID 19) were synthesised by JPT Peptide Technologies GmbH. The lyophilised peptides were resuspended in 100 μl of DMSO and subsequently diluted down to a concentration of 1 mg/ml with 10 mM PBS pH7.4, snap frozen and stored at −80° C. in aliquots.

The magnetic particles were activated by 2 washes with 1 ml 0.1M MES buffer pH 5.0 using the MagnetoPure (Chemicell, Berlin, Germany) magnetic separator. After the second wash the particles were resuspended in 0.25 ml 0.1M MES buffer pH5.0. The carboxyl groups on the green fluorescent magnetic beads nano-screenMAG/G-ARA were activated with freshly prepared EDC (1-Ethyl-3-(3-dimethylaminopropyl) carbodiimide) (Sigma) by dissolving 10 mg EDC in 0.25 ml 0.1M MES buffer. The EDC buffer was added to the particles and gently mixed at RT for 10 min. Following this incubation, the magnetic particles were washed 2× with 1 ml 0.1M MES buffer pH5.0 and resuspended in 0.25 ml of 0.1M MES buffer, pH5.0.

200 μg of scFv#6 purified protein were mixed with 150 μg of Tat cell penetration peptide and 200 μg of Gaussia luciferase export peptide in 0.25 ml of 0.1M MES buffer, pH 5.0. The protein mixture was added to 10 mg of activated particles and gently mixed for two hours at room temperature to generate fluorescent peptide complexes. The particles were washed 3× with 1 ml PBS and resuspended in 500 μl Blocking/Storage buffer (10 mM PBS pH7.4, 0.1% BSA, 0.05% sodium azide).

Fully protected Tat-cell penetrating peptide, Acetyl-GRKKRRQRRRPQ (SEQ ID 11), and fully protected Gaussia Luciferase secretory peptide, Acetyl-MGVKVLFALICIAVAEA (SEQ ID 19), were synthesised by Spheritech Ltd by solid phase synthesis and cleaved from the resin to leave a free carboxyl group at the C-terminus and all other functional groups protected.

The peptides (200 ug of each) were redissolved in DMF and activated by the addition of EDC and N-hydroxysuccinimide and this solution of activated peptides was mixed with 10 mg of nano-screenMAG-PEA beads and gently mixed for 2 hours at room temperature. The particles were washed three times with 1 ml of DMF and then resuspended in 1 ml of Trifluoroacetic acid (TFA) and gently mixed for 2 hours at room temperature. The particles were washed with 5×1 ml of water and 3×1 ml of 0.1M MES buffer, pH 5.0, then resuspended in 0.25 ml of 0.1M MES buffer, pH 5.0. 200 ug of scfv#6 purified protein were mixed with 0.25 ml of EDC buffer, added to the particles and gently mixed at room temperature for two hours. The particles were washed 3× with 1 ml PBS and resuspended in 500 μl Blocking/Storage buffer (10 mM PBS pH7.4, 0.1% BSA, 0.05% sodium azide).

Preparation of Quantum Dot/Antibody/Peptide Conjugates

Quantum dot (QD), cell penetrating peptide, secretory peptide and antibody conjugates were produced. Amine-functionalized quantum dots, purchased from Nanoco Technologies Ltd., Manchester), were activated by adding approximately 1 mg of sulfo-SMCC to 200 μl of amine quantum dots (QD514; n=2.3 nmol) in 200 μl of 50 mM sodium phosphate pH 7.4. Tat-cell penetrating peptide, Acetyl-GRKKRRQRRRPQC (SEQ ID 20), and fully protected Gaussia Luciferase secretory peptide, Acetyl-MGVKVLFALICIAVAEAC (SEQ ID 21), were synthesised by Spheritech Ltd leaving a free cysteine sulfhydryl at the C-terminus of each peptide. The antibody and two different peptides were mixed at the same molarity prior to incubation with the maleimide-activated quantum dot in conjugation buffer (1 mM EDTA, 0.1 M phosphate, 0.15 M NaCl, pH 7.2) and incubated at 4° C. overnight.

After conjugation, the quantum dot-antibody/peptide conjugates were centrifuged at 90,000 rpm for 3 hours, and the pellet was dissolved in a PBS (Phosphate Buffered Saline) solution. Quantum dot antibody/peptide conjugates with a QD to peptide molar ratio of 1:100 up to 1:300 were prepared.

Acid functionalised quantum dots, purchased from Nanoco Technologies Ltd (Manchester) were activated with freshly prepared EDC (1-Ethyl-3-(3-dimethylaminopropyl) carbodiimide) (Sigma) by dissolving 10 mg EDC in 0.25 ml 0.1M MES buffer. The EDC buffer was added to the particles and gently mixed at RT for 10 min.

200 μg of scFv#6 purified protein were mixed with 150 μg of Tat cell penetrating peptide and 200 μg of Gaussia luciferase export peptide in 0.25 ml of 0.1M MES buffer, pH 5.0. The protein mixture was added to activated QD and gently mixed for two hours at room temperature to generate fluorescent peptide complexes.

After conjugation, the quantum dot-antibody/peptide conjugates were centrifuged at 90,000 rpm for 3 hours, and the pellet was dissolved in a PBS (Phosphate Buffered Saline) solution. Quantum dot antibody/peptide conjugates with a QD to peptide molar ratio of 1:100 up to 1:300 were prepared.

Cell Culture

Chinese Hamster Ovary (CHO) cells and the A549 human lung carcinoma cell line (which constitutively express mutant K-RasG12V antigen) were maintained in selective Gibco® Ham's F12 Nutrient Mixture Media (Life Technologies) supplemented with 10% heat-inactivated foetal bovine serum, 100 μg/mL streptomycin, 0.29 mg of L-glutamine at 37° C. in a humidified atmosphere of 5% CO₂.

Intracellular Cell Imaging of a Native Intracellular Target Protein

The CHO and A549 cell lines were seeded the day before LCI, at 2×10⁵ cells/well in a 24 well glass bottom plate (In Vitro Scientific) in 1 ml of growth media to allow the cells to reach 90-95% confluency at the time of LCI. The following day the media was replaced with fresh growth media pre-warmed at 37° C. A serial dilution ranging between 40 μg and 2.4 μg of the magnetic-protein mix was gently mixed with 100 μl of serum free Ham's F12 media and added drop wise to the cells. The cells were incubated with the magnetic-protein complexes for 1.30 h at 37° C. in a humidified atmosphere of 5% CO₂ to allow these to transduce into the cells. The cells were then vigorously washed five times with PBS and incubated further for 3 h. Following the 3 incubation, any unbound magnetic-protein complexes are secreted from the cells through the cell secretory pathway. The cells were then vigorously washed five times with PBS and viewed under a confocal microscope.

Image Acquisition Laser Scanning Confocal Microscopy.

Laser scanning confocal microscopy was carried out using an Inverted Zeiss LSM 510 META Axiovert 200M confocal microscope using a 10×, 20× and a 40×/1.3NA oil immersion objective. FITC was imaged using an Argon 488-nm laser light and a 505-530-nm BP emission filter.

### EXAMPLE 1

A method to perform live cell imaging (LCI) on native intracellular proteins was developed by coupling fluorescent magnetic particles to scFv antibody fragments and to cell penetrating and cell export peptides (FIG. 1). Cell penetrating peptides have enjoyed a huge success at delivering various proteins and particles into cells. Magnetic beads have successfully been coupled to cationic peptides (Smith et al., 2010) and shown to enhance their uptake in vivo. In particular the Tat and antennapedia cell penetrating peptides have shown very good uptake profiles in CHO, A549 and HeLa cell lines, displaying similar kinetic update profiles in all three cell lines (Jones et al., 2005). However, no study, to date, has previously reported the usage of a cell export peptide to facilitate the export of a nanoparticle from a cell. In this study the proto-oncogene mutant RasG12V was used as a model system to test the LCI technology. Mutations in the Ras protein occur in approximately 30% of human cancers, with higher frequencies in pancreas, colon and lung adenocarcinoma (Grewal et al., 2011). The Ras protein belongs to the family of proteins known as GTPases, and it is a very important key player in signal transduction as molecular switches. The activity of the Ras protein is mediated through two switch regions displaying conformational differences between active (GTP bound) and inactive (GDP bound) states (Vetter and Wittinghofer, 2001). Occasionally, mutations can occur in all three Ras protein isoforms (H-Ras, N-Ras and K-Ras) resulting in constitutively activated GTP-bound forms that promote cell transformation in a signal-independent manner (Adjei, 2001). This then activates cell proliferation and the anti-apoptotic Ras signalling cascade.

### EXAMPLE 2

A scFv antibody fragment, the scFv#6 antibody, was employed as a ligand specific for the target RasG12V protein. The scFv#6 was shown in previous studies (Tanaka et al., EMBO Journal 2007) to bind specifically to the activated form of mutant RasG12V and to block the activity of this protein. The gene for this antibody was synthesised by GeneArt (Life Technologies) and following expression in bacteria and affinity purification, it was subsequently coupled to green fluorescent magnetic nanoparticles together with the Tat penetrating peptide and the Gaussia luciferase export peptide. The cell penetrating peptide was used to facilitate the transport of the complexes into cells. The cell export peptide was used to direct the fluorescent nanoparticles to the extracellular milieu if the target RasG12V protein was not present (FIG. 1). A human lung carcinoma cell line, A549 cells, constitutively expressing RasG12V was used to exemplify the technique. The Chinese Hamster Ovary (CHO) cell line, expressing normal levels of activated Ras but not mutant RasG12V, was used as a negative control. The cells were incubated in the presence of the fluorescent nanoparticles for a period of 1.30 h allowing the beads to internalise into the cells via peptide-mediated transduction. The cells were extensively washed with PBS to remove any excess of beads, and then further incubated for another 3 h to allow the beads to follow the secretory pathway if they did not anchor to the RasG12V proteins. The cells were then vigorously washed again with PBS before visualization with a confocal microscope (FIG. 2).

The Ras isoforms (H-Ras, N-Ras and K-Ras) are mainly localised to the inner cell membrane, however, in recent years they have been shown to associate, as well, with intracellular organelles such as the Golgi, the ER, endosomes and the mitochondria (Grewal et al., 2011). In this study the fluorescent scFv magnetic nanoparticles localised to the cell membrane, to the perinuclear membrane and to distinct subcellular vesicles in the cytoplasm consistent with the localisation pattern of RasG12V in the A549 lung cancer cell line (FIG. 2). The cell export peptide reduced considerably the background of the nanoparticles accumulating non-specifically in the cytoplasm due to the non-expression of the target protein RasG12V in the CHO cells.

It is not clear how the export peptide interacts with the cell secretory pathway and is able to secrete these fluorescent nanoparticles. The export peptide could be either following a classical or an unconventional cell secretory pathway (Nickel, 2005). Further studies are underway to determine the mechanism.

A simple method is described here for performing LCI on native intracellular proteins and thereby removing the need for lengthy and complex cloning steps. Furthermore, it solves the background problem observed when using nanoparticles such as QDs on native intracellular proteins. If the QD does not bind to its target it will simply accumulate in the cytoplasm.

### EXAMPLE 3

Cells were incubated with beads for 30 min, washed (t=0 min) and analysed by FACS, or further incubated for 10, 30, 60, 120 or 180 mins before being analysed by FACS. Beads functionalised with poly dimethylamine were used as a positive control for cell entry (100% of entry). Samples were washed every 30 min. CHO cells comprising beads with the tat and export peptide (closed diamond) retain the beads for longer. 24.3% of the beads comprising the Tat peptide alone (closed diamond) were retained in the cells after 120 min incubation, whereas 13.2% beads comprising both, the Tat and exit peptides (closed triangle), were retained in the cells after the two hour incubation period (FIG. 4).

The method is fast and simple, and requires minimal work. Furthermore, no cellular cytotoxicity was observed allowing the cells to be used for other downstream applications.

TABLE 1
Intracellular Cancer stem cell markers

| | | |
|---|---|---|
| TP53INP11 | tumor protein p53 inducible | ENSG00000164938 |
| | nuclear protein 1 | |
| Axl | AXL receptor tyrosine kinase | ENSG00000167601 |
| GATA-3 | GATA binding protein 3 | ENSG00000107485 |
| USP22 | ubiquitin specific peptidase 22 | ENSG00000124422 |
| BIRC6 | baculoviral IAP repeat containing 6 | ENSG00000115760 |
| Hh | sonic hedgehog | ENSG00000164690 |

TABLE 2
Transcription factors and the consensus sequences
of DNA or RNA binding elements (P refers to
purine, Y to pyrimidine, N is any nucleotide)

| SEQ ID | name | Sequence |
|---|---|---|
| 24 | p53 | AGACATGCCTN₍₀₋₁₃₎AGACATGCCT |
| 25 | TEF-1 | CTGGAATGT |
| 26 | CREB | TGACGTCA |
| 27 | RUNX3 | PYGPYGGT |
| 28 | NF-KB | GGGRNYY YCC |
| 29 | YAP-1 | T(T/G)ACTAA |
| 30 | HUR | AUUUA, |
| 31 | HUR | AUUUUA |
| 32 | HUR | AUUUUUA |
| 33 | HNF3/FOX | AATAACT |
| 34 | Hox-1.3 | CYYNATTA(T/G)Y |
| 35 | STAT | TTCNNGAA |
| 36 | STAT | TTCNNNGAA |
| 37 | STAT | TTCNNNNGAA |

### REFERENCES

1. Adjei A A (2001) Blocking oncogenic Ras signaling for cancer therapy. J Natl Cancer Inst 93: 1062-1074.
2. Alivisators P. (1996) Semiconductor clusters, nanocrystals, and quantum dots. Science 271:933-7.
3. Avignolo C, Bagnasco L, Biasotti B., Melchiori A., Tomati V., Bauer I., Sails A., Chiossone L, Mingari M. C, Orecchia P., Carnemoila B., Neri D., Zardi L., Parodi S. (2008) Internalization via Antennapedia protein transduction domain of an scFv antibody toward c-Myc protein. The FASEB Journal 22:1237-1245
4. Bacia K, Kim S A, Schwille P. (2006) Fluorescence cross-correlation spectroscopy in living cells. Nat Methods 3:83-89
5. Chan W, Maxwell D J, Gao X H, Bailey R E, Han M Y, Nie S M. (2002) Luminescent quantum dots for multiplexed biological detection and imaging. Curr Opin Biotechnol 13:40-6.
6. Chen H, Bai C, Wang X (2010) Application of Probes in Live Cell Imaging. Journal of Epithelial Biology & Pharmacology 3:40-48
7. Cho S, Jang J, Song C, Lee H, Ganesan P, Yoon T Y, Kim M W, Choi M C, Ihee H, Heo W D, Park Y K. (2013) Simple super-resolution live-cell imaging based on diffusion-assisted Forster resonance energy transfer. Scientific Reports 3:1208-1215
8. Chudakov D M, Matz M V, Lukyanov S, Lukyanov K A (2010) Fluorescent Proteins and Their Applications in Imaging Living Cells and Tissues. Physiol Rev 90:1103-1163
9. Futaki S., Suzuki T., Ohashi W., Yagami T., Tanaka S., Ueda K., Sugiura Y. Arginine-rich peptides. (2001) An abundant source of membrane-permeable peptides having potential as carriers for intracellular protein delivery. J Biol chem. 276 (8): 5836-5840
10. Gao X H, Cui Y Y, Levenson R M, Chung L, Nie S M. (2004) In vivo cancer targeting and imaging with semiconductor quantum dots. Nat Biotechnol 22:969-76.
11. Grewal T., Koese M., Tebar F., Enrich C. (2011) Differential Regulation of RasGAPs in Cancer. Genes & Cancer 2(3):288-297
12. Jones S W, Christison R, Bundell K, Voyce C J, Brockbank S M V, Newham P, Lindsay M A. (2005) Characterisation of cell-penetrating peptide-mediated peptide delivery. British Journal of Pharmacology 145:1093-1102
13. Kerppola T K. (2006) Visualization of molecular interactions by fluorescence complementation. Nat Rev Mol Cell Biol 7:449-456
14. Liu D S, Tangpeerachaikul A, Selvaraj R, Taylor M T, Fox J M, Ting A Y. (2012) Diels-Alder Cycloaddition for Fluorophore Targeting to Specific Proteins inside Living Cells. J Am Chem Soc. 134(2):792-795
15. Liu W, Howarth M, Greytak A B, Zheng Y, Nocera D G, Ting A Y, Bawendi M G. (2008) Compact Biocompatible Quantum Dots Functionalized for Cellular Imaging. J. Am. Chem. Soc. 130:1274-1284
16. Ma D X, Shi N Q, Qi X R. (2011) Distinct transduction modes of arginine-rich cell-penetrating peptides for cargo delivery into tumor cells. Int J Pharm.
17. Mao S, Benninger R K, Yan Y, et al. (2008) Optical lock-in detection of FRET using synthetic and genetically encoded optical switches. Biophys J 94:4515-24.
18. Michalet X, Pinaud F F, Bentolila L A, Tsay J M, Doose S, Li J J, Sundaresan G, Wu A M, Gambhir S S, Weiss S. (2005) Quantum Dots for Live Cells, in Vivo Imaging, and Diagnostics. Science. 307(5709):538-544
19. Nickel W. (2005) Unconventional Secretory Routes: Direct Protein Export Across the Plasma Membrane of Mammalian Cells. Traffic 6:607-614
20. Niesner U., Halin C, Lozzi L., Gunthert M., Neri P., Wunderli-Allenspach H., Zardi L., and Neri D. (2002) Quantitation of the Tumor-Targeting Properties of Antibody Fragments Conjugated to Cell-Permeating HIV-1 TAT Peptides. Bioconjugate Chem. 13(4):729-736
21. Patterson G H. (2007) Fluorescent proteins for cell biology. Methods Mol Biol 411:47-80.
22. Rhyner M N, Smith A M, Gao X, Mao H, Yang L, Nie S. (2008) Quantum dots and targeted nanoparticle probes for in vivo tumor imaging. Fundan Biomed Technol 102:413-425.
23. Smith C A, de la Fuente J, Pelaz B, Furlani E P, Mullin M, Berry C C. (2010) The effect of static magnetic fields and tat peptides on cellular and nuclear uptake of magnetic nanoparticles. Biomaterials 31(15):4392-400
24. Tanaka T, Williams R L, Rabbitts T H (2007) Tumour prevention by a single antibody domain targeting the interaction of signal transduction proteins with RAS. EMBO J. 26(13): 3250-3259
25. Tokuko H. (2002) Live cell imaging: approaches for studying protein dynamics in living cells. Cell Struct Fund 27:333-4.
26. Treanor B, Lanigan P M, Suhling K, et al. (2005) Imaging fluorescence lifetime heterogeneity applied to GFP-tagged MHC protein at an immunological synapse. J Microsc 217:36-43.
27. Tsien R. Y. (1998) The green fluorescent protein. Annu Rev Biochem 67:509-44.
28. Uttamapinant C, White K A, Baruah H, Thompson S, Fernández-Suárez M, Puthenveeti S, Ting A Y. (2010) A fluorophore ligase for site-specific protein labeling inside living cells. PNAS 107(24): 10914-10919
29. Vetter I R, Wittinghofer A (2001) The guanine nucleotide-binding switch in three dimensions. Science 294: 1299-1304.
30. Walling M A, Novak J A, Shepard J R E. (2009) Quantum Dots for Live Cell and In Vivo Imaging. Int. J. Mol. Sci. 10:441-491
31. Watkins R W, Lavis L D, Kungb V M, Los G V, Raines R T. (2009) Fluorogenic affinity label for the facile, rapid imaging of proteins in live cells. Org Biomol Chem. 7(19):3969-3975
32. Wombacher R & Cornish V W (2011) Chemical tags: applications in live cell fluorescence imaging. J. Biophotonics 4(6): 391-402
33. Zipfel W R, Williams R M, Webb W W. (2003) Nonlinear magic: multiphoton microscopy in the biosciences. Nat Biotechnol 21:1369-77.
34. O. Cochef, M. Kenigsberg, I. Delumeau, M. Duchesne, F. Schweighoffer, B. Tocque, J-L. Teillaud, Intracellular expression and functional properties of an anti-p21Ras scFv derived from a rat hybridoma containing specific A and irrelevant κ light chains. Molecular immunology 35 (1998): 1097-1110

1. A fluorescent bead comprising:
a ligand that specifically binds an intracellular antigen,
a cell membrane penetrating cationic peptide, and
a peptide comprising an amino acid sequence that is adapted to interact with the secretory pathway in a cell.

2. The bead according to claim 1, wherein said fluorescent bead is selected from the group consisting of: magnetic particles, semiconductor nanoparticles (NPs), quantum dots, metal NPs, and silica NPs.

3.-4. (canceled)

5. The bead of claim 1, wherein said fluorescent bead comprises two or more fluorescent molecules, wherein said fluorescent molecules have different excitation/emission wavelengths.

6. The bead according to claim 1, wherein said intracellular antigen is substantially a nuclear antigen.

7. The bead according to claim 6 wherein said nuclear antigen is a FOX family members, TARP, p53, Rb, E2F, hK4, BRCA1, Cdk2, SATB1, TP53INP1, Myc, Fos, Jun, CREB, Ets, SRF, FAK, Pax6, Calpain, Elk1, Stats 1-3, Akt, or p21.

8. The bead according to claim 1, wherein said intracellular antigen is substantially a cytoplasmic antigen.

9. The bead according to claim 8, wherein said cytoplasmic antigen is Jak1, Jak2, Tyk2, Jak3, GATA 1-4, Stats 1-6, CBP, NFkB, IKK, PIK3CA, B-raf, EBI3, eIF2α, Akt, PI3K, IAP, Hsp, FAK, Raf, Ras, TNF, Src, Abl, or Caspases 1-12.

10. The bead according to claim 1, wherein said cell membrane penetrating cationic peptide is a natural cell penetrating peptide.

11. The bead according to claim 1, wherein said cell membrane penetrating cationic peptide is a synthetic sequence.

12. The bead according to claim 1, wherein said cell membrane penetrating cationic peptide is adapted to penetrate a eukaryotic cell.

13. The bead according to claim 12, wherein said eukaryotic cell is a mammalian cell.

14. The bead according to claim 12, wherein said cell membrane penetrating cationic peptide is YARKKRRQRRR (SEQ ID NO: 1), YARKARRQARR (SEQ ID NO: 2), YARAAARQARA (SEQ ID NO: 3), YARAARRAARR (SEQ ID NO:4), YARAARRAARA (SEQ ID NO: 5), YARRRRRRRRR (SEQ ID NO: 6), YAAARRRRRRR (SEQ ID NO: 7), PLSSIFSRIGDP (SEQ ID NO: 8), RQIKIWFQNRRMKWKK (SEQ ID NO: 9), WEIEDEDER (SEQ ID NO: 10), GRKKRRQRRRPQ (SEQ ID NO: 11), RRRRRRRRRRRR (SEQ ID NO: 12), or GRKKRRQRRRPQC (SEQ ID NO: 20).

15.-16. (canceled)

17. The bead according to claim 1, wherein the cell membrane penetrating cationic peptide and the peptide adapted to interact with the secretory pathway is engrafted into the CDR3 regions of the VL and VH domains.

18. The bead according to claim 1, wherein the cell membrane penetrating cationic peptide is a single peptide comprising a cell penetrating domain and a domain adapted to remove fluorescent beads that are not bound to said intracellular antigen.

19. The bead according to claim 18, wherein said single peptide comprises the amino acid motif RXRR (SEQ ID NO: 38) or RXKR (SEQ ID NO: 39) where X is any amino acid residue.

20. The bead according to claim 1, wherein said peptide adapted to interact with the secretory pathway is
(SEQ ID NO: 13)
MCPARSLLLVATLVLLDHLSLA;
(SEQ ID NO: 14)
MDAMKRGLCCVLLLCGAVFVSPS;
(SEQ ID NO: 15)
MATGSRTSLLLAFGLLCLPWLQEGSA;
(SEQ ID NO: 16)
MYRMQLLSCIALSLALVTNS;
(SEQ ID NO: 17)
MGVKVLFALICIAVAE;
(SEQ ID: 19)
MGVKVLFALICIAVAEA;
or
(SEQ ID NO: 21)
MGVKVLFALICIAVAEAC.

21. The bead according to claim 20, wherein said secretory pathway is a mammalian secretory pathway.

22. The bead according to claim 1, wherein said bead further comprises a nuclear export peptide.

23. The bead according to claim 22, wherein said nuclear export peptide comprises the amino acid sequence:
[SEQ ID NO: 18]
NELALKLAGLDINKTEGEEDAQ.

24. The bead according to claim 1, wherein said ligand that binds said intracellular antigen comprises an antibody fragment.

25. (canceled)

26. The bead according to claim 1, wherein said ligand that binds said intracellular antigen is a peptide, peptide aptamer, or nucleic acid aptamer.

27. The bead according to claim 1, wherein said intracellular antigen is a transcription factor and said ligand is a nucleic acid comprising a nucleotide binding motif for said transcription factor.

28. The bead according to claim 27 wherein said transcription factor is TEF-1, CREB, RUNX3, NFκB, YAP-1, HUR, HNF3 [FOXA], Hox-1.3, STAT, or p53.

29. The bead according to claim 27, wherein said nucleotide binding motif is a sequence set forth in SEQ ID: NO 24, 25, 26, 27, 28, 29, 30, 33, 34, 35, 36 or 37.

30.-36. (canceled)

37. A method to image live eukaryotic cells, comprising:
i) contacting a sample comprising live eukaryotic cells with the fluorescent bead of claim 1;
ii) allowing uptake of said fluorescent bead by the cell and binding to an intracellular target; and
iii) applying electromagnetic radiation of a defined wavelength to detect the intracellular target thereby imaging said cell.

38. The method according to claim 37 wherein said eukaryotic cells are mammalian cells.

39. The method according to claim 38 wherein said mammalian cells are human cells.

40. The method according to claim 37, wherein said cells are stem cells.

41. The method according to claim 40, wherein said stem cells are pluripotent stem cells, multipotent stem cells, or lineage restricted stem cells.


SEQUENCE LISTING (39 sequences)

SEQ ID NO: 1 - protein, 11 aa, Artificial Sequence
  cell membrane penetrating peptide
YARKKRRQRRR

SEQ ID NO: 2 - protein, 11 aa, Artificial Sequence
  cell membrane penetrating peptide
YARKARRQARR

SEQ ID NO: 3 - protein, 11 aa, Artificial Sequence
  cell membrane penetrating peptide
YARAAARQARA

SEQ ID NO: 4 - protein, 11 aa, Artificial Sequence
  cell membrane penetrating peptide
YARAARRAARR

SEQ ID NO: 5 - protein, 11 aa, Artificial Sequence
  cell membrane penetrating peptide
YARAARRAARA

SEQ ID NO: 6 - protein, 11 aa, Artificial Sequence
  cell membrane penetrating peptide
YARRRRRRRRR

SEQ ID NO: 7 - protein, 11 aa, Artificial Sequence
  cell membrane penetrating peptide
YAAARRRRRRR

SEQ ID NO: 8 - protein, 12 aa, Artificial Sequence
  cell membrane penetrating peptide
PLSSIFSRIGDP

SEQ ID NO: 9 - protein, 16 aa, Artificial Sequence
  cell membrane penetrating peptide
RQIKIWFQNRRMKWKK

SEQ ID NO: 10 - protein, 9 aa, Artificial Sequence
  Cell membrane penetrating peptide
WEIEDEDER

SEQ ID NO: 11 - protein, 12 aa, Artificial Sequence
  cell membrane penetrating peptide
GRKKRRQRRRPQ

SEQ ID NO: 12 - protein, 12 aa, Artificial Sequence
  cell membrane penetrating peptide
RRRRRRRRRRRR

SEQ ID NO: 13 - protein, 22 aa, Artificial Sequence
  secretory peptide
MCPARSLLLVATLVLLDHLSLA

SEQ ID NO: 14 - protein, 23 aa, Artificial Sequence
  secretory peptide
MDAMKRGLCCVLLLCGAVFVSPS

SEQ ID NO: 15 - protein, 26 aa, Artificial Sequence
  secretory peptide
MATGSRTSLLLAFGLLCLPWLQEGSA

SEQ ID NO: 16 - protein, 20 aa, Artificial Sequence
  secretory peptide
MYRMQLLSCIALSLALVTNS

SEQ ID NO: 17 - protein, 16 aa, Artificial Sequence
  secretory peptide
MGVKVLFALICIAVAE

SEQ ID NO: 18 - protein, 22 aa, Artificial Sequence
  Nuclear export peptide
NELALKLAGLDINKTEGEEDAQ

SEQ ID NO: 19 - protein, 17 aa, Artificial Sequence
  Gaussia sceretory peptide
MGVKVLFALICIAVAEA

SEQ ID NO: 20 - protein, 13 aa, Artificial Sequence
  cell penetrating peptide
GRKKRRQRRRPQC

SEQ ID NO: 21 - protein, 18 aa, Artificial Sequence
  Gaussia Luciferase secretory peptide
MGVKVLFALICIAVAEAC

SEQ ID NO: 22 - DNA, 868 bp, Homo sapiens
gcggccgcaggaggtactcacgatgaaatatttactgccgacagccgcagccggcctgttactgttagcagcacagccggcgatggcgcaggtacagttacaggaaagcggtggcggtctggttcagcctggccgtagtctgaagctgagctgtgaagccagtggtttcaccgtgagcaactacggcatggcctggattcgccaggcccctaccaaggggctggagtgggtggccagtatcagctacgacggcagcagtacctactaccgcgatagtgtgaagggccgcttcacaatcagtcgcgacaacggcaaaagcaccctgtacctgcagatggacagtctgcgtagtgaggacaccgccacctactattgtgcccgccctaaggtgccgggctacaacctgaattggttcgcctattggggccagggcaacaccgtgaccgtgagcagtggtggtggcggtagtggtggtggtggtagtggcggcggcagtcagttcgtgttaacccagcctaccagcgtgagtctgagcctgggcagcaccgtgaaactgagctgtaccctgagcagcggcaacatcgagaacagctacgtgcactggtatcagcaatacgagggtcgtagcccgaccaccatgatttacaacgacgacaaacgcccggatggcgttccggaccgcttcagtggcagcattgacagcagcagcaatagcgccttcctgaccatcaacaacgtggagatcgaggacgaggccatctacttctgccacagctatgtgagcagcatccacatcttcggtggcggcaccaaactgaccgtgctggacgatgatgacaagcaccatcaccatcaccactaatctaga

SEQ ID NO: 23 - protein, 279 aa, Homo sapiens
MKYLLPTAAAGLLLLAAQPAMAQVQLQESGGGLVQPGRSLKLSCEASGFTVSNYGMAWIRQAPTKGLEWVASISYDGSSTYYRDSVKGRFTISRDNGKSTLYLQMDSLRSEDTATYYCARPKVPGYNLNWFAYWGQGNTVTVSSGGGGSGGGGSGGGSQFVLTQPTSVSLSLGSTVKLSCTLSSGNIENSYVHWYQQYEGRSPTTMIYNDDKRPDGVPDRFSGSIDSSSNSAFLTINNVEIEDEAIYFCHSYVSSIHIFGGGTKLTVLDDDDKHHHHHH

SEQ ID NO: 24 - DNA, 21 bp, artificial sequence
  consensus sequences of DNA or RNA binding elements
agacatgcctnagacatgcct

SEQ ID NO: 25 - DNA, 9 bp, artificial sequence
  consensus sequences of DNA or RNA binding elements
ctggaatgt

SEQ ID NO: 26 - DNA, 8 bp, artificial sequence
  consensus sequences of DNA or RNA binding elements
tgacgtca

SEQ ID NO: 27 - DNA, 8 bp, artificial sequence
  consensus sequences of DNA or RNA binding elements
rygryggt

SEQ ID NO: 28 - DNA, 10 bp, artificial sequence
  consensus sequences of DNA or RNA binding elements
gggrnyyycc

SEQ ID NO: 29 - DNA, 7 bp, artificial sequence
  consensus sequences of DNA or RNA binding elements
tnactaa

SEQ ID NO: 30 - DNA, 5 bp, artificial sequence
  consensus sequences of DNA or RNA binding elements
auuua

SEQ ID NO: 31 - DNA, 6 bp, artificial sequence
  consensus sequences of DNA or RNA binding elements
auuuua

SEQ ID NO: 32 - DNA, 7 bp, artificial sequence
  consensus sequences of DNA or RNA binding elements
auuuuua

SEQ ID NO: 33 - DNA, 6 bp, artificial sequence
  consensus sequences of DNA or RNA binding elements
ataact

SEQ ID NO: 34 - DNA, 10 bp, artificial sequence
  consensus sequences of DNA or RNA binding elements
cyynattany

SEQ ID NO: 35 - DNA, 8 bp, Artificial Sequence
  consensus sequences of DNA or RNA binding elements
ttcnngaa

SEQ ID NO: 36 - DNA, 9 bp, artificial sequence
  consensus sequences of DNA or RNA binding elements
ttcnnngaa

SEQ ID NO: 37 - DNA, 10 bp, artificial sequence
  consensus sequences of DNA or RNA binding elements
ttcnnnngaa

SEQ ID NO: 38 - protein, 4 aa, Artificial Sequence
  motif RXRR
RXRR

SEQ ID NO: 39 - protein, 4 aa, Artificial Sequence
  motif RXKR
RXKR